(19) United States
(12) Patent Application Publication
(10) Pub. No.: US 2026/0047819 A1
(43) Pub. Date: Feb. 19, 2026

(54) SYSTEMS AND METHODS FOR CONFIGURING AN ULTRASOUND IMAGING DEVICE TO BE TRACKABLE

(71) Applicant: Clarius Mobile Health Corp., Vancouver (CA)

(72) Inventors: Kris Dickie, Vancouver (CA); Laurent Pelissier, North Vancouver (CA)

(21) Appl. No.: 18/807,360

(22) Filed: Aug. 16, 2024

(51) Int. Cl. A61B 8/00

(52) U.S. Cl. CPC A61B 8/4263; A61B 8/54; A61B 8/56

(57) ABSTRACT

A method for configuring an ultrasound imaging device to be trackable with short-range wireless connection during an active mode and an inactive mode comprises during the active mode, applying one or more data signals being communicated by the short-range wireless connection for location tracking, and while in the inactive mode, intermittently engaging the short-range wireless connection for initiating location tracking of the ultrasound imaging device.

### FIELD OF THE INVENTION

The present disclosure relates generally to ultrasound imaging, and in particular, systems and methods for configuring an ultrasound imaging device to be trackable.

### BACKGROUND OF THE INVENTION

Ultrasound imaging systems are an important tool for diagnosis and therapy in a wide range of medical applications. Conventionally, ultrasound imaging systems were large, expensive units used only in radiology departments by highly trained specialists. To improve portability and usability and enable ultrasound to be used at the point-of-care and by more users, various attempts have been made to reduce the size and cost of these systems and avoid the ergonomically troublesome cables that are typically used to attach handheld transducers to processing hardware.

In part to facilitate the reduction in size of these systems and to increase mobility, ultrasound imaging systems can include a mobile device that communicates with an ultrasound imaging device via a wireless connection. Often in such systems, the ultrasound imaging device can be misplaced due to its wireless feature.

For example, wireless ultrasound imaging devices can be left in pockets of clothing and/or left on tables, desks, and/or medical carts. This is particularly problematic in larger institutions, such as hospitals, where there are often numerous users of the wireless ultrasound imaging devices and numerous locations in which a wireless ultrasound imaging device can be misplaced. Furthermore, once the remaining battery life of a misplaced wireless ultrasound imaging device drains, the misplaced wireless ultrasound imaging device cannot be located electronically.

Tracking the location of a wireless ultrasound imaging device can be challenging. For example, in some locations such as within a building, standard tracking technology such as Global Positioning System (GPS) is not suitable or operational. Other technology may be used for location tracking, such as Bluetooth™. However, enabling the activation or triggering of Bluetooth™ while the ultrasound imaging device is in an inactive mode presents further challenges. Furthermore, tracking the location of a wireless ultrasound imaging device with limited battery power requires balanced power management to ensure that the battery power lasts long enough to locate the device.

There is a need for ultrasound imaging systems that can be configured to be trackable in both an active mode and an inactive mode.

The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

Non-limiting examples of various embodiments of the present disclosure will next be described in relation to the drawings, in which:

FIG. 1 is a schematic diagram of a system with multiple ultrasound scanners, according to an embodiment of the present invention;

FIG. 2 is a schematic diagram of an ultrasound imaging system, according to an embodiment of the present invention;

FIG. 3 is a flowchart diagram showing steps of a method for configuring an ultrasound imaging device to be trackable, in accordance with at least one embodiment of the present invention;

FIG. 4A is a flowchart diagram showing additional steps of the method of FIG. 3, in accordance with at least one embodiment of the present invention;

FIG. 4B is a flowchart diagram showing additional steps of the method of FIG. 3, in accordance with at least one embodiment of the present invention;

FIG. 4C is a flowchart diagram showing additional steps of the method of FIG. 3, in accordance with at least one embodiment of the present invention;

FIG. 5 is an illustration of an ultrasound imaging device, according to an embodiment of the present invention; and

FIG. 6 is a schematic diagram of an ultrasound imaging device locator system, according to an embodiment of the present invention.

Unless otherwise specifically noted, articles depicted in the drawings are not necessarily drawn to scale.

### DETAILED DESCRIPTION OF THE INVENTION

### A. Glossary

The term “communications network” and “network” can include both a mobile network and data network without limiting the term's meaning, and includes the use of wireless (e.g. 2G, 3G, 4G, 5G, WiFi®, WiMAX®, Wireless USB (Universal Serial Bus), Zigbee®, Bluetooth® and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “module” can refer to any component in this invention and to any or all of the features of the invention without limitation. A module may be a software, firmware or hardware module (or part thereof), and may be located or operated within, for example, in the ultrasound scanner, a display device or a server.

The term “multi-purpose electronic device” or “display device” or “computing device” or “off-the-shelf display computing device” or “mobile device” is intended to have broad meaning and includes devices with a processor communicatively operable with a screen interface, for example, such as, laptop computer, a tablet computer, a desktop computer, a smart phone, a smart watch, spectacles with a built-in display, a television, a bespoke display or any other display device that is capable of being communicably connected to an ultrasound scanner. Such a device may be communicatively operable with an ultrasound scanner and/or a cloud-based server (for example via one or more communications networks). Such device may be combined with processor, non-transitory memory, and/or user input device in a shared electronic device, or there may be peripheral display devices which may comprise a monitor, touchscreen, projector, or other display device known in the art, which may enable a user to view ultrasound images produced by an ultrasound imaging system, and/or interact with various data stored in non-transitory memory.

The term “operator” (or “user”) may (without limitation) refer to the person that is operating an ultrasound scanner (for example, a clinician, medical personnel, a sonographer trainer, a student, a vet, a sonographer/ultrasonographer and/or ultrasound technician). This list is non-exhaustive.

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as in cloud computing or local virtualization, which are ultimately grounded in physical processors.

The term “system” when used herein, and not otherwise qualified, may include an ultrasound scanner and a multi-purpose electronic device/display device; and/or an ultrasound scanner, multi-purpose electronic device/display device and a server. The system may include one or more applications operating on a multi-purpose electronic device/display device to which the ultrasound scanner is communicatively connected.

The term “ultrasound transducer” (or “probe” or “ultrasound probe” or “transducer” or “ultrasound scanner” or “scanner” or “ultrasound imaging device”) refers to a wide variety of transducer types including but not limited to linear transducer, curved transducers, curvilinear transducers, convex transducers, microconvex transducers, and endocavity probes. In operation, an ultrasound scanner is often communicatively connected to a multi-purpose electronic device/display device to direct operations of the ultrasound scanner, optionally through one or more applications on the multi-purpose electronic device/display device (for example, via the Clarius™ App).

The term “workflow application” or “application” (for example, via the Clarius™ App) or “workflow” refers to a software tool that assists with the automated activation and/or configuration of device feature(s). Conveyance to an operator may be visually on the display screen or via audio.

### B. Exemplary Embodiments

In a first broad aspect of the present disclosure, there is provided a method for configuring an ultrasound imaging device to be trackable with short-range wireless connection during an active mode and an inactive mode, the method including: during the active mode, applying one or more data signals being communicated by the short-range wireless connection for location tracking; and while in the inactive mode, intermittently engaging the short-range wireless connection for initiating location tracking of the ultrasound imaging device.

In some embodiments, intermittently engaging the short-range wireless connection to initiate location tracking of the ultrasound imaging device while in the inactive mode includes: detecting a movement at the ultrasound imaging device; and in response to detecting the movement, activating the short-range wireless connection to initiate location tracking during the inactive mode.

In some embodiments, detecting the movement at the ultrasound imaging device includes receiving a motion signal from a motion sensor at the ultrasound imaging device, the motion signal being indicative of the ultrasound imaging device moving away from a receiving device.

In some embodiments, the ultrasound imaging device moves in recognizable patterns in the active mode and detecting the movement at the ultrasound imaging device includes receiving a motion signal from a motion sensor at the ultrasound imaging device, the motion signal being indicative of the ultrasound imaging device moving in a non-recognizable pattern.

In some embodiments, detecting the movement at the ultrasound imaging device includes: receiving a motion signal from a motion sensor at the ultrasound imaging device; comparing the movement at the ultrasound imaging device to a set of pre-determined movements which correspond to the active mode, for initiating location tracking of the ultrasound imaging device; and if the movement at the ultrasound imaging device does not match at least one of the pre-determined movements, activating the short-range wireless connection to initiate location tracking.

In some embodiments, intermittently engaging the short-range wireless connection to initiate location tracking of the ultrasound imaging device while in the inactive mode includes intermittently operating a tracking trigger at the ultrasound imaging device to activate the short-range wireless connection to initiate location tracking while the ultrasound imaging device is in the inactive mode.

In some embodiments, intermittently engaging the short-range wireless connection to initiate location tracking of the ultrasound imaging device while in the inactive mode includes prioritizing a portion of battery power at the ultrasound imaging device for activating the short-range wireless connection for initiating location tracking while the ultrasound imaging device is in the inactive mode.

In some embodiments, the method further includes transmitting one or more beacon signals for initiating location tracking of the ultrasound imaging device.

In some embodiments, the method further includes transmitting the one or more beacon signals to a workflow application at a mobile device.

In some embodiments, the method further includes transmitting one or more beacon signals for initiating location tracking of the ultrasound imaging device through at least one of an Apple™ and an Android™ finding protocol.

In some embodiments, the short-range wireless connection includes one of a Bluetooth™ and a Zigbee™ connection.

In some embodiments, the short-range wireless connection includes a Bluetooth™ connection which additionally applies one or more data signals to pair the ultrasound imaging device with a mobile device.

In some embodiments, the active mode includes an operating mode during which the ultrasound imaging device is in use.

In some embodiments, the inactive mode includes one or more operating modes during which the ultrasound imaging device is in one of hibernation or sleep.

In another broad aspect of the present disclosure, there is provided an ultrasound imaging device configurable to be trackable with short-range wireless connection during an active mode and an inactive mode, the ultrasound imaging device including: a short-range wireless connection module; and a processor in communication with the short-range wireless connection module. The processor is operable to: during the active mode, apply one or more data signals being communicated by the short-range wireless connection module for location tracking; and during the inactive mode, intermittently engage the short-range wireless connection module for initiating location tracking of the ultrasound imaging device.

In some embodiments, the processor is further operable to: detect a movement at the ultrasound imaging device; and in response to detecting the movement, activate the short-range wireless connection module to initiate location tracking during the inactive mode.

In some embodiments, the ultrasound imaging device includes a motion sensor and the processor is further operable to receive a motion signal from the motion sensor, the motion signal being indicative of one or more of: the ultrasound imaging device moving away from a receiving device; the ultrasound imaging device moving in a non-recognizable pattern, wherein the ultrasound imaging device moves in recognizable patterns in the active mode; and the movement being non-matching to a set of pre-determined movements which correspond to the active mode, wherein the movement at the ultrasound imaging device is compared to the set of pre-determined movements, for initiating location tracking of the ultrasound imaging device, and if the movement at the ultrasound imaging device does not match at least one of the pre-determined movements, the short-range wireless connection module is activated to initiate location tracking.

In some embodiments, the processor is further operable to: intermittently operate a tracking trigger of the ultrasound imaging device to activate the short-range wireless connection module to initiate location tracking while the ultrasound imaging device is in the inactive mode.

In some embodiments, the processor is further operable to prioritize a portion of battery power at the ultrasound imaging device for activating the short-range wireless connection module for initiating location tracking while the ultrasound imaging device is in the inactive mode.

In another broad aspect of the present disclosure, there is provided a non-transitory computer readable media for configuring an ultrasound imaging device to be trackable with short-range wireless connection during an active mode and an inactive mode, the media including computer-readable instructions, which, when executed by one or more processors on the ultrasound imaging device, configures the ultrasound imaging device to: during the active mode, apply one or more data signals being communicated by the short-range wireless connection for location tracking; and while in the inactive mode, intermittently engaging the short-range wireless connection for initiating location tracking of the ultrasound imaging device.

The system and method of the present invention enables configuration of an ultrasound imaging device to be trackable via short-range wireless connection. In one aspect, location tracking via short-range wireless connection enables location tracking of an ultrasound imaging device while the ultrasound imaging device is in one of various possible operational states.

There are increasing commercial advantages in portable, handheld, wireless ultrasound imaging devices, often referred to as POCUS (point of care ultrasound) devices, which are often the size of a smartphone. Such devices can often be misplaced due to the devices' wireless feature. The method and system of the invention provide a configuration tool which is particularly useful in the configuration of POCUS devices for location tracking. Such imaging devices use a transducer (e.g., a piezoelectric or capacitive device operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer.

Ultrasound imaging devices may become misplaced, particularly when the ultrasound imaging devices are wireless. Ultrasound imaging devices may periodically advertise their presence. For example, a communication unit of the ultrasound imaging device may include a Bluetooth™ Low Energy module that is configured to periodically advertise the presence of the ultrasound imaging device. That is, while an ultrasound imaging device is in an active mode, data signals that can be used for location tracking, such as Bluetooth™ signals, may be active. However, in some instances, an ultrasound imaging device may not advertise its presence (e.g., location tracking is not available) during the active mode for privacy reasons. Furthermore, while the ultrasound imaging device is in an inactive mode, data signals that can be used for location tracking are generally reduced or non-existent. With the disclosed embodiments, the ultrasound imaging device can be configured to be trackable while in an active mode and an inactive mode.

Some consumer tracking devices operate using technologies such as Bluetooth™ for tracking objects for extended periods of time (e.g., years) without requiring a battery change due to low power requirements of the device. However, medical devices, such as ultrasound imaging devices often have significant power consumption requirements, which can make location tracking of such devices challenging. The disclosed embodiments provide power management that enables the ultrasound imaging device to be configured to be trackable while in an active mode and an inactive mode.

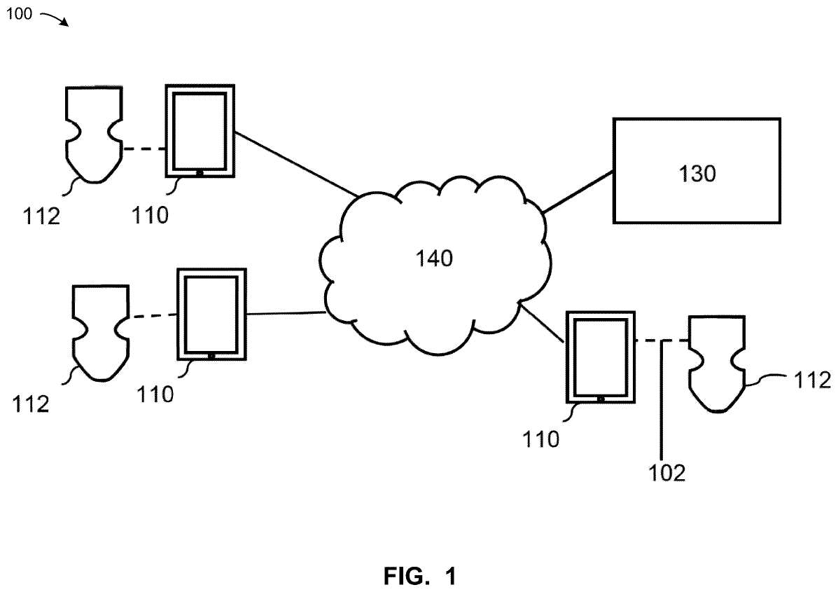

FIG. 1 is a schematic diagram of a system 100 with multiple ultrasound imaging devices 112 in accordance with an embodiment of the disclosure. In the system 100 shown, there are multiple ultrasound imaging devices 112 connected to their corresponding mobile devices 110 and either connected directly, or indirectly via the mobile devices 110, to a communications network 140, such as the internet. One or more of the ultrasound imaging devices 112 and mobile devices 110 shown in FIG. 1 may be the same or different as the other.

Generally, the ultrasound imaging devices 112 may have any of a wide range of various sizes and configurations. For example, the ultrasound imaging device 112 may be handheld or hand carried. In some preferred embodiments, the ultrasound imaging device 112 may have the form of hand-held battery-powered probes.

The ultrasound imaging devices 112 may be connected via the communications network 140 to a server 130. The server 130 may include a processor, which may be connected to a non-transitory computer readable memory storing computer readable instructions, which, when executed by the processor, cause the server to provide one or more of the functions of the system 100. The server 130 may be a cloud-based server configured to communicate with the mobile devices 110 via a workflow application installed thereon, or in another fashion. The ultrasound imaging devices 112 may be connected to the mobile devices 110 via link 102 which is described further below as link 202.

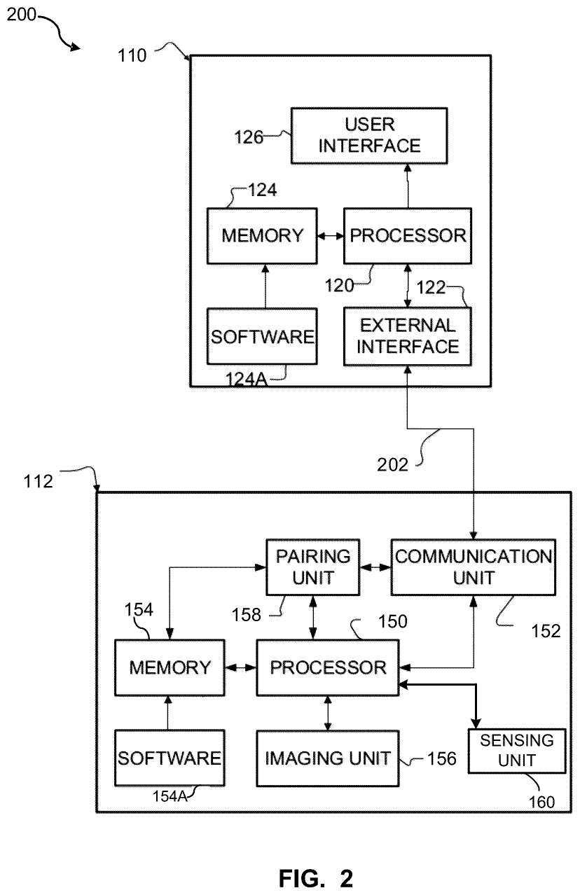

FIG. 2 is a schematic diagram of an example system 200 composed of an example mobile device 110 in communication with an example ultrasound imaging device 112.

The ultrasound imaging device 112 may be wirelessly connected with the mobile device 110. The ultrasound imaging device 112 may transmit an ultrasound signal to a target object according to a control signal that is transmitted from the mobile device 110. A communication link 202 between the mobile device 110 and the ultrasound imaging device 112 may be established. The mobile device 110 may gather information about the ultrasound imaging device 112 by way of link 202. The mobile device 110 may establish communication link 202 with one or more other ultrasound imaging devices 112 and may obtain and use information about the ultrasound imaging devices 112 for other purposes.

The mobile device 110 may comprise a processor 120, memory 124, user interface 126, and an external interface 122. The processor 120 may be a general purpose central processing unit (CPU) or may be a low power/mobile specific processor. The processor 120 is coupled with the memory 124. The memory 124 includes storage for program and program operating code. One or more programs 124A in memory 124 coordinates interactions of the mobile device 110 with ultrasound imaging devices 112 as described herein. In some embodiments, the memory 124 may further store motion data, such as motion data related to one or more recognizable scanning patterns and/or pre-determined movements, which may be retrieved by processor 120.

The user interface 126 is coupled with processor 120 and may comprise both the software and hardware components necessary to interface with a user of the mobile device 110. The user interface 126 may comprise physical input devices such as a touch sensitive display screen, keyboard, microphone, or function buttons. The user interface 126 may further comprise output devices such as a color, grayscale, or black and white display screen, audio speaker/output, vibrating or LED indicators.

The external interface 122 is coupled with the processor 120 and provides connectivity of the mobile device 110 with the ultrasound imaging device(s) 112 though communication link(s) 202. The external interface 122 may also be operable to communicate with another device, such as a web server.

The processor 120 may generate control signals to control an operation of ultrasound imaging device 112 according to information that is provided via the user interface 126. The control signals may include control signals that control ultrasound imaging device 112 to generate ultrasound signals, and control signals that control how ultrasound imaging device 112 handles transmission and reception of the ultrasound signal. The processor 120 may control wireless communication with the ultrasound imaging device 112, and may control generation and display of an ultrasound image on a display of user interface 126 based on ultrasound image data provided from ultrasound imaging device 112. In addition, and as described further below, the processor 120 may receive and process inputs relating to motion of ultrasound imaging device 112. For example, processor 120 may receive data from processor 150 via communication unit 152, such data being acquired from sensing unit 160.

The ultrasound imaging device 112 may comprise a processor 150, memory 154 (storing software 154A), imaging unit 156, pairing unit 158, communication unit 152, and sensing unit 160. The processor 150 may comprise a general-purpose CPU, a low power/mobile specific processor, a field programmable gate array (FPGA), a combination of two or more of these or the like.

The imaging unit 156 is operable to acquire ultrasound image data of a target object based on control signals from processor 150. The imaging unit 156 may comprise a transmitter for generating ultrasound energy and a receiver for receiving ultrasound energy reflected from the target object. The imaging unit 156 may further comprise an analog-to-digital converter for digitizing the received ultrasound energy into digital ultrasound data. The imaging unit 156 may further comprise one or more beamformers to combine and focus the received ultrasound energy along a desired scanline. The imaging unit 156 may further comprise a signal processor to apply filtering or compression to the ultrasound image data. The imaging unit 156 may also comprise a scan converter for converting the ultrasound image data into a specific display format.

The processor 150 is coupled with memory 154. The memory 154 includes storage for program and program operating code. One or more programs in memory 154 coordinates the operation of the ultrasound imaging device 112 as described herein. The memory 154 may also be used to store information about ultrasound imaging device 112 and/or ultrasound image data. The memory 154 can include a non-transitory computer readable memory for storing computer readable instructions, which, when executed by the processor 150, may cause the ultrasound imaging device 112 to provide one or more of the functions of the system 200. Such functions may be, for example, the acquisition of ultrasound data, the processing of ultrasound data, the scan conversion of ultrasound data, the transmission of ultrasound data or ultrasound frames to a display device, the detection of operator inputs to the ultrasound scanner, and/or the switching of the settings of the ultrasound scanner.

The pairing unit 158 is operable to establish the communication link 202 between the communication unit 152 and the external interface 122 of the mobile device 110. The communication unit 152 may comprise one or more wireless transceivers.

The communication unit 152 may comprise a short-range wireless connection module in communication with processor 150 for providing short-range wireless connection capabilities. For example, the short-range wireless connection module can generally provide short-range wireless connection capabilities in the range of approximately 1 to 100 meters, depending on technology. The short-range wireless connection capabilities can be used for location tracking of the ultrasound imaging device 112. In some embodiments, the short-range wireless connection includes one of a Bluetooth™ and a Zigbee™ connection. For example, the Bluetooth™ connection may include a Bluetooth™ low energy (BLE) connection. Alternatively, one or more of the following protocols may be used: wireless local area network (LAN), Wi-Fi, Wi-Fi Direct (WFD), ultra wideband (UWB), infrared data association (IrDA), near field communication (NFC), wireless broadband internet (Wibro), world interoperability for microwave access (WiMAX), shared wireless access protocol (SWAP), radio frequency (RF) communications and the like.

The communication unit 152 may comprise an integrated circuit that provides both Bluetooth™ and Wi-Fi provisioning. For example, the integrated circuit may be multi-use. The integrated circuit may provide discovery and/or locating capabilities using Bluetooth™ provisioning. The integrated circuit may further provide pairing capabilities, which allows the ultrasound imaging device 112 to connect with the mobile device 110 using Wi-Fi provisioning (which may be a higher power use). That is, the integrated circuit that provides Bluetooth™ provisioning may further include integrated provisioning for Wi-Fi, without requiring a separate component. In some embodiments, data that can be applied for location tracking may be communicated via Bluetooth™ and/or Wi-Fi provisioning.

The communication link 202 may comprise more than one communication protocol. The protocol used for communications between the ultrasound imaging device 112 and the mobile device 110 may be Wi-Fi, Bluetooth™ or Zigbee™, for example, or any other suitable two-way radio communications protocol. In some embodiments, the ultrasound imaging device 112 may operate as a WiFi™ hotspot, for example. In some embodiments, the communication link 202 may be wired. For example, the ultrasound imaging device 112 may be attached to a cord that may be pluggable into a physical port of the the mobile device 110.

The sensing unit 160 may comprise one or more sensors operable to measure one or more parameters. In some embodiments, the sensing unit 160 comprises one or more inertial measurement unit (IMU) sensors for measuring movement of the ultrasound imaging device 112. For example, movement of the ultrasound imaging device 112 can be measured based on the linear acceleration, angular velocity, and/or magnetic field strength of the ultrasound imaging device 112. In some embodiments, the sensing unit 160 comprises one or more accelerometer, gyroscope, and/or magnetometer, or any other conventionally known or future developed motion sensor for measuring movement of the ultrasound imaging device 112.

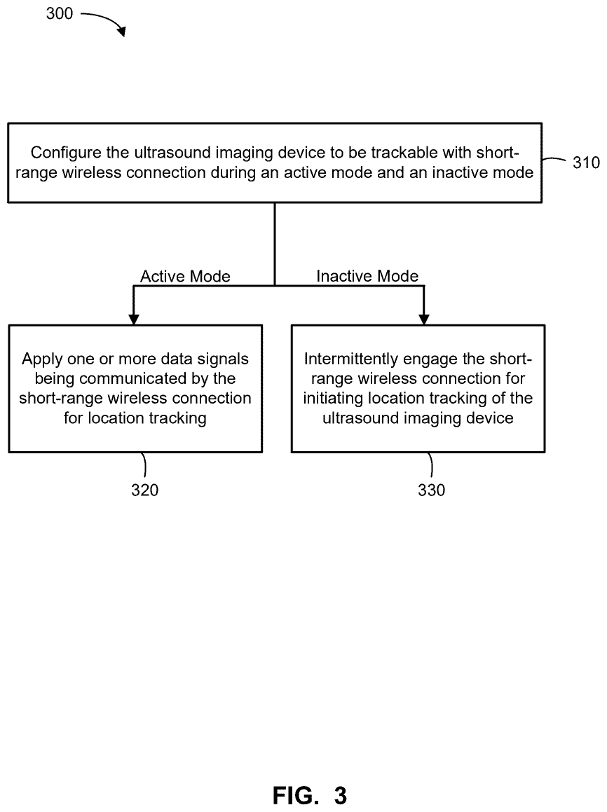

Reference will now be made to FIG. 3, which is a flowchart diagram of a method 300 provided in accordance with one embodiment of the invention for configuring the ultrasound imaging device 112.

At step 310, the ultrasound imaging device 112 is configured to be trackable with short-range wireless connection during an active mode and an inactive mode. That is, the ultrasound imaging device 112 can be configured to be trackable even while the ultrasound imaging device 112 is in an inactive mode.

The short-range wireless connection can be provided via the short-range wireless connection module, as described herein. For example, the short-range wireless connection can include one of a Bluetooth™ and a Zigbee™ connection, although the present invention is not intended to be limited to any one type of connection. In some embodiments, the short-range wireless connection includes a Bluetooth™ connection which additionally applies one or more data signals to pair the ultrasound imaging device 112 with the mobile device 110.

The various components of the ultrasound imaging device 112 may be capable of operating in several different power modes, such as an active mode and an inactive mode, in which each of the different power modes consume power at different power levels. These different power modes may be achieved by modifying the operation of software, hardware, or some combination of both software and hardware. For example, a hardware-based power mode change may involve changing a given component from a normal operation mode to a low-power operation mode, or powering off the component completely. A software-based power mode change may involve disabling a power-intensive processing step, or reducing the number of ultrasound frames in a particular time period (e.g., reducing frame rate).

Based on the operating condition of the ultrasound imaging device 112, the ultrasound imaging device 112 may reduce power consumption by directing selected components to operate at different modes. In various embodiments, the ultrasound imaging device 112 may be configured to operate in a number of modes, each with a respective power level.

The active mode may include an operating mode during which the ultrasound imaging device 112 is in use. For example, the active mode may include instances during which the ultrasound imaging device 112 is powered on and is being used to acquire ultrasound images. In some embodiments, during the active mode, various power-intensive imaging parameters or features (e.g., higher frame rate, spatial compounding, multi-line acquisition) may be enabled so that optimal images are acquired. The active mode may include instances during which the ultrasound imaging device 112 is powered on and is idle before, during, and/or after acquiring ultrasound images, for example, while positioning a patient for acquiring the ultrasound images. The active mode may additionally include instances in which a user is not acquiring ultrasound images but instead is engaging with a mobile device 110 to select settings to be used on the powered on ultrasound imaging device 112, to view previously acquired images on the mobile device 110 and/or to attach or detach peripheral devices (such as for example a cooling fan) on or from a powered on ultrasound imaging device 112. Active mode may also be broadly viewed as any instance in which the FPGA processor, such as processor 150, within an ultrasound imaging device 112, is in a high powered state.

An inactive state may be broadly viewed as any instance in which the FPGA processor, such as processor 150, within an ultrasound imaging device 112 is off. In some embodiments, the microcontroller of the ultrasound imaging device 112 may remain in an “on” or active orientation. The inactive mode may include one or more operating modes during which the ultrasound imaging device 112 is not being used or set up to be used for imaging, such as hibernation or sleep modes. Hibernation and sleep modes are generally lower power operating modes than the active mode. Hibernation mode is generally a lower power mode than sleep mode. Hibernation mode may include instances in which the ultrasound imaging device 112 is powered down but retains its system state. For example, the system state may be retained in non-transitory computer readable memory of memory 154 during hibernation mode. The system state may include, for example, contents of volatile computer readable memory of memory 154, running processes on processor 150, and/or system variables. Sleep mode may include instances in which the ultrasound imaging device 112 is in low-power operation in which the processing functions of the ultrasound imaging device 112 are reduced to a minimal. For example, during sleep mode, the ultrasound imaging device 112 may continue to consume power in order to maintain the system state in memory 154 but reduces processing functions by suspending computational tasks. That is, during sleep mode, the contents of volatile computer readable memory of memory 154 may be maintained in the volatile computer readable memory. The inactive mode may include any other operating mode (e.g., hybrid sleep, standby, etc.) during which the ultrasound imaging device 112 is similarly in a low power operational state and/or is configured for minimal processing.

At step 320, during the active mode, the processor 150 applies one or more data signals being communicated by the short-range wireless connection for location tracking.

The data signal being communicated by the short-range wireless connection can be any suitable data signal. In some embodiments, the data signal being communicated by the short-range wireless connection is a location data signal, such as a Bluetooth™ data signal. However, the data signal being communicated by the short-range wireless connection does not necessarily need to be a location data signal. For example, during the active mode, the ultrasound imaging device 112 may be in wireless communication with the mobile device 110 (e.g., via communication link 202), and various data signals may be communicated by the ultrasound imaging device 112 via the wireless connection, which may be used for location tracking. That is, by virtue of the wireless connection between the ultrasound imaging device 112 and the mobile device 110, the processor 120 may determine that the ultrasound imaging device 112 is in the active mode, and accordingly may trigger certain communication for location tracking. For example, the processor 120 may trigger data communication from other components of the ultrasound imaging device 112 (e.g., GPS data) for obtaining location and/or tracking information of the ultrasound imaging device 112.

At step 330, during the inactive mode, the processor 150 can intermittently engage the short-range wireless connection for initiating location tracking of the ultrasound imaging device 112. In some embodiments, the intermittent time period between engaging the short-range wireless connection is variable. In some embodiments, the intermittent time period between engaging the short-range wireless connection is regular or non-variable. In some embodiments, the intermittent time period may be based on the remaining battery power of the ultrasound imaging device 112. For example, the intermittent time period may be larger (i.e., less frequent engagement of the short-range wireless connection) when the remaining battery power of the ultrasound imaging device 112 is lower, and the intermittent time period may be higher (i.e., more frequent engagement of the short-range wireless connection) when the remaining battery power of the ultrasound imaging device 112 is higher. In some examples, the length of the intermittent time period may depend on the power consumption of the short-range wireless (such as Bluetooth) advertisement. In some other examples, the intermittent time period may vary based on the previously detected location of the ultrasound imaging device 112. It may be that based on that previously detected location, the intermittent time period is shorter as there is a greater need for active location tracking over other locations. In some embodiments, the intermittent time period can be selected by a user, for example, via the user interface 126. In some embodiments, the intermittent time period can range generally from 1 second to 60 seconds, more particularly from 1 second to 30 seconds and even more particularly from 1 second to 10 seconds. The present invention is not intended to be limited to any specific intermittent ranges, as improvements to the power consumption of short-range wireless advertising will likely improve over time with various advancements in technologies and/or other factors.

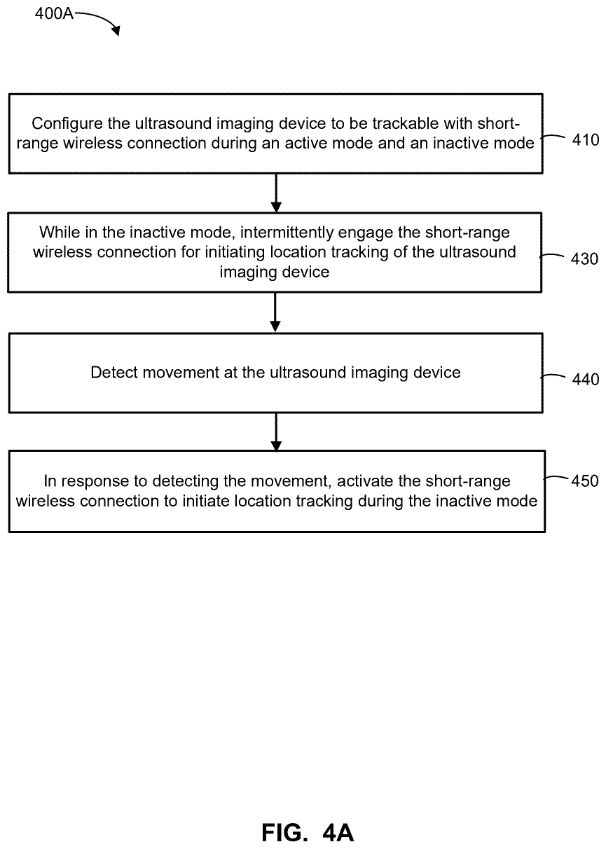

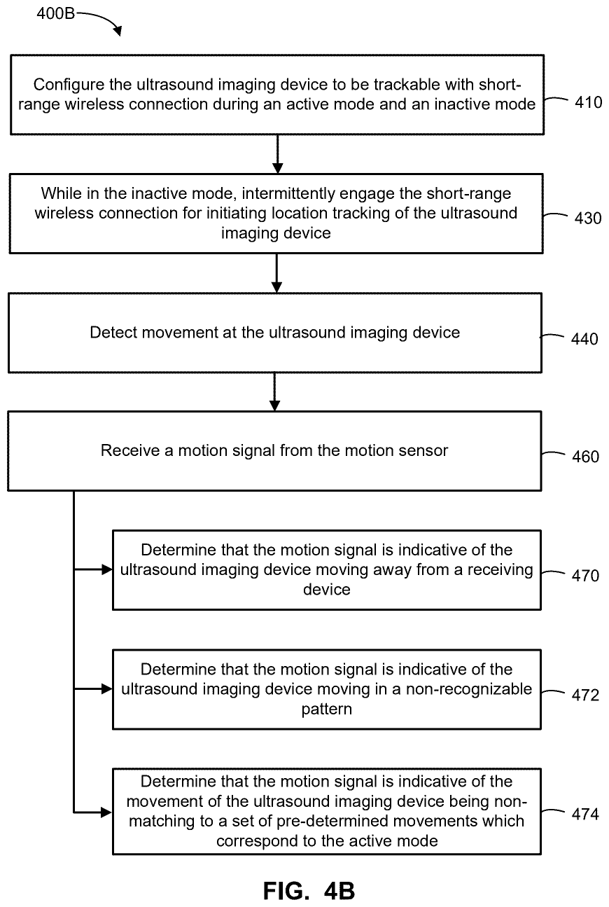

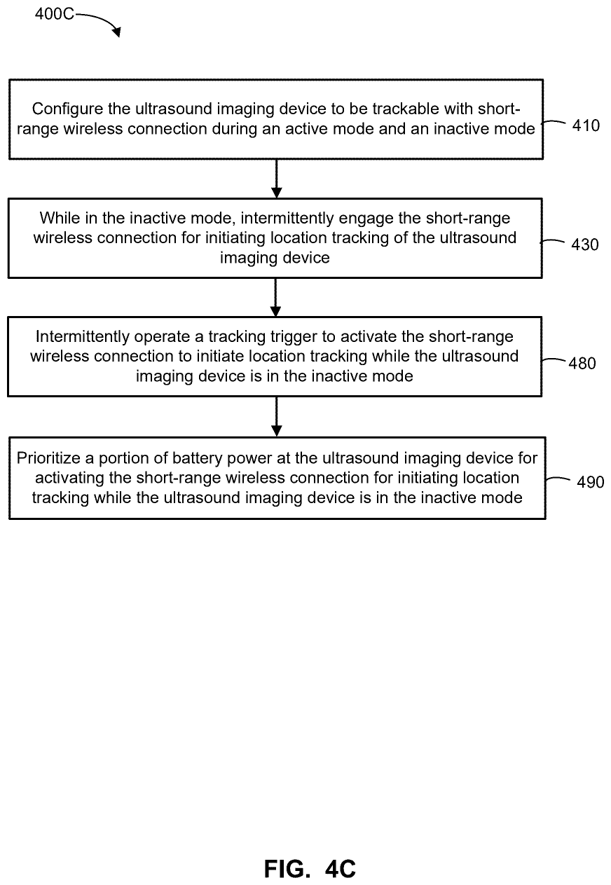

Reference will now be made to FIGS. 4A-C, each of which shows a flowchart diagram of methods 400A to 400C, respectively, provided in accordance with embodiments of the invention.

Referring to FIG. 4A, at step 410, the ultrasound imaging device 112 is configured to be trackable with short-range wireless connection during an active mode and an inactive mode. Step 410 may be analogous to step 310.

At step 430, during the inactive mode, the short-range wireless connection is intermittently engaged for initiating location tracking of the ultrasound imaging device 112. Step 430 may be analogous to step 330. Step 430 may include steps 440 and 450, each of which will described in further detail.

At step 440, movement is detected at the ultrasound imaging device 112. Movement can include, for example, scanning movement (e.g., while the ultrasound imaging device 112 is being used to acquire ultrasound images), preparation movement (e.g., while the ultrasound imaging device 112 is being prepared and/or positioned before and/or after acquiring ultrasound images), and/or transportation movement (e.g., while the ultrasound imaging device 112 is being transported, such as being carried by a user or moved on a medical cart).

Referring to FIG. 4B, step 440 may include step 460. At step 460, a motion signal is received from a motion sensor at the ultrasound imaging device 112. The motion signal may be received at the processor 120. The motion sensor may be one of the one or more sensors of the sensing unit 160. For example, processor 120 may receive data from processor 150 via communication unit 152, such data being acquired from sensing unit 160. In some embodiments of the invention, the motion signal may include data measured from an inertial measurement unit. In some embodiments, the motion signal includes position data, velocity data, linear acceleration data, orientation data, angular velocity data, angular acceleration data, and/or magnetic field strength data. For example, instantaneous and/or interval-based measurements may be used. In some embodiments, the motion signal is received in a raw, unprocessed format. In alternative embodiments, the motion signal is received in a preprocessed format.

In some embodiments, processing of the motion signal is based on a power state of the ultrasound imaging device 112. For example, the processor 120 may process the motion signal as described herein when the ultrasound imaging device 112 is in a low power state (i.e., when short-range wireless connection may otherwise be inactive by default). In such embodiments, the processor 120 may not process the motion signal while the ultrasound imaging device 112 is in a full power state.

At step 470, the processor 120 determines that the motion signal is indicative of the ultrasound imaging device 112 moving away from a receiving device. In some embodiments, the receiving device may be the mobile device 110. In alternative embodiments, the receiving device may be a docking device, such as a charging station, or a beacon associated or communicably connected with the ultrasound imaging device 112.

The processor 120 may determine that the motion signal is indicative of the ultrasound imaging device 112 moving away from a receiving device based on an increasing distance between the ultrasound imaging device 112 and the receiving device. For example, the motion signal may include a linear acceleration of the ultrasound imaging device 112. The processor 120 may determine from the linear acceleration data that the distance between the ultrasound imaging device 112 and the receiving device is increasing. In some embodiments, both the ultrasound imaging device 112 and the receiving device may be moving. In such embodiments, the processor 120 may determine that the motion signal is indicative of the net movement of the ultrasound imaging device 112 and the receiving device being such that the ultrasound imaging device 112 is moving away from the receiving device.

As an alternative to motion sensor, a receiving device may employ a Received Signal Strength Indicator (RSSI) to determine the distance between an ultrasound imaging device 112 and the receiving device. RSSI is a measurement of the power present in a received radio signal and is indicated by a negative dBm value, wherein a higher number, is indicative of a better signal and hence closer proximity between the ultrasound imaging device 112 and the receiving device. A decrease in signal strength is an indicator of a decreasing proximity between the ultrasound imaging device 112 and the receiving device.

The ultrasound imaging device 112 may be moving away from the receiving device in a variety of manners. For example, the ultrasound imaging device 112 may be carried by a person, carried in a pocket of an article of clothing, or moved on or within another object, such as a medical cart.

At 472, the processor 120 determines that the motion signal is indicative of the ultrasound imaging device 112 moving in a non-recognizable pattern. Non-recognizable patterns can include, for example, movement of the ultrasound imaging device 112 away from a receiving device (as discussed above), movements that are faster than a recognizable speed of movement during the active mode, and/or movements that are bigger than a recognizable size of movements during the active mode.

In some embodiments, sensing unit 160 may acquire speed data of the ultrasound imaging device 112. The processor 120 may compare the speed of movement of the ultrasound imaging device 112 to speed data of recognizable patterns of movement. Movement of the ultrasound imaging device 112 that is faster than a defined limit of the recognizable pattern of movement may be indicative of a “transport” movement rather than a scanning motion of the ultrasound imaging device 112.

In some embodiments, sensing unit 160 may acquire data of the ultrasound imaging device 112 that can indicate the size of a movement of the ultrasound imaging device 112. The processor 120 may compare the size of movement of the ultrasound imaging device 112 to size data of recognizable patterns of movement. Movement of the ultrasound imaging device 112 that is bigger than a defined limit of the recognizable pattern of movement may be indicative of a “transport” movement rather than a scanning motion of the ultrasound imaging device 112.

In some embodiments, whether or not the motion signal is indicative of one or more recognizable patterns may be based on an imaging pre-set. That is, certain ultrasound imaging device 112 motion signals may indicate an absence of operator activity for a type of imaging pre-set, but the same motion signals may indicate normal imaging activity for another type of imaging pre-set.

One or more motion signals that are indicative of one or more of recognizable patterns may be stored in memory 124. The processor 120 may be operable to compare the received motion signal from the motion sensor at the ultrasound imaging device 112 to the one or more stored motion signals that are indicative of the recognizable patterns. Based on this comparison, the processor 120 may determine that the motion signal is indicative of the ultrasound imaging device 112 moving in a non-recognizable pattern.

Returning to FIG. 4B, at 474, the processor 120 determines that the motion signal is indicative of the movement of the ultrasound imaging device 112 being non-matching to a set of pre-determined movements which correspond to the active mode. For example, the processor 120 may compare the movement at the ultrasound imaging device 112 to a set of pre-determined movements which correspond to the active mode, for initiating location tracking of the ultrasound imaging device 112. If the movement at the ultrasound imaging device 112 does not match at least one of the pre-determined movements, the processor 120 may activate the short-range wireless connection to initiate location tracking.

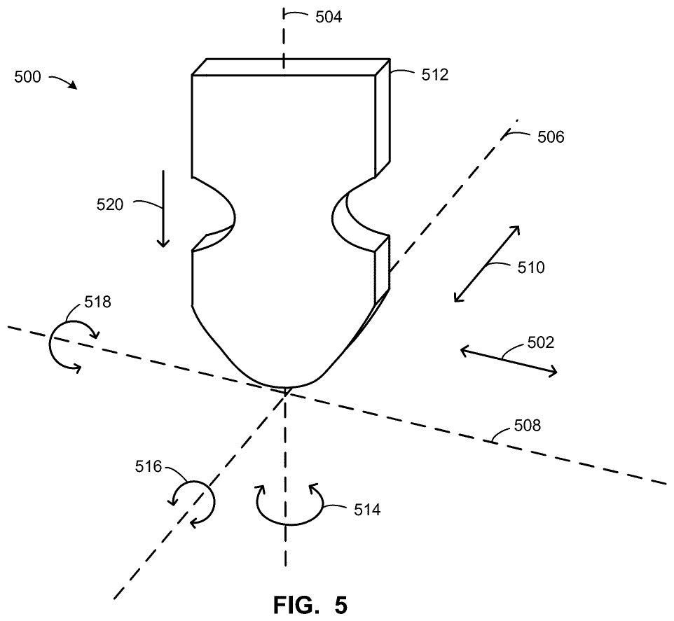

Referring to FIG. 5, shown therein is an illustration of an example ultrasound imaging device 512, which may be analogous to ultrasound imaging device 112. As shown in FIG. 5, ultrasound imaging device 512 may move in a variety of pre-determined movements in the active mode. Pre-determined movements may comprise a plurality of basic movements that are performed when scanning with the ultrasound imaging device 512 and include, but are not limited to: sweep, slide, rock, tilt (or fan), rock, rotate, and compression. Ultrasound imaging device 512 may move in a sweeping movement 510 along axis 506. Ultrasound imaging device 512 may move in a sliding movement 502 along axis 508. Sweeping and sliding involve moving the entire ultrasound probe on the subject, in one or more specific directions, to find a desired imaging window. This movement may be employed to find the best window, move to different areas of the subject body, or to follow the course of a specific structure (such as a vessel). Ultrasound imaging device 512 may move in a tilting movement 518 about axis 508. Tilting (or fanning) the ultrasound probe involves moving the transducer from side to side along the short axis of the probe. This specific pre-determined movement allows visualization of multiple cross-sectional images of a structure of interest. Ultrasound imaging device 512 may move in a rotating movement 514 about axis 504. Rotating the ultrasound probe involves turning the transducer in a clockwise or counterclockwise direction along its central axis. Rotation is most commonly used to switch between the long and short axis of a specific structure such as a vessel, the heart, the kidney, etc. Ultrasound imaging device 512 may move in a rocking movement 516 about axis 506. Rocking the ultrasound probe involves a movement of the ultrasound probe either towards or away from the probe indicator along the long-axis and allows centering of a region or interest. This is also referred to as “in-plane” motion because the image is kept in-plane throughout this manipulation. Finally, ultrasound imaging device 512 may move in a compression movement 520 along axis 504. Compression with the ultrasound probe involves putting downward pressure on the probe to evaluate the compressibility of a structure or organ of interest. In some embodiments, ultrasound imaging device 512 may move using one or more pre-determine movements 510, 512, 514, 516, 518, and 520.

Returning now to FIG. 4A, at step 450, in response to detecting the movement at step 440, the short-range wireless connection is activated to initiate location tracking during the inactive mode. As discussed herein, in some embodiments, activating the short-range wireless connection to initiate location tracking during the inactive mode can involve activating a Bluetooth™ module for transmitting a Bluetooth™ data signal.

Referring to FIG. 4C, at step 480, intermittently engaging the short-range wireless connection to initiate location tracking of the ultrasound imaging device 112 while in the inactive mode includes intermittently operating a tracking trigger at the ultrasound imaging device 112 to activate the short-range wireless connection to initiate location tracking while the ultrasound imaging device 112 is in the inactive mode.

In some embodiments, the ultrasound imaging device 112 includes the tracking trigger. For example, the tracking trigger may be a hardware component of the ultrasound imaging device 112. In some embodiments, the tracking trigger may be implemented through software, such as software 154A. The tracking trigger may provide an electrical means of intermittent activation of the short-range wireless connection while the ultrasound imaging device 112 is in a low power state, such as an inactive mode. The tracking trigger enables location tracking of the ultrasound imaging device 112 to be triggered by a temporal protocol, particularly while the ultrasound imaging device 112 is in an inactive mode. In some embodiments, the intermittent time period between operating tracking trigger is variable. In alternative embodiments, the intermittent time period between operating the tracking trigger is regular or non-variable. In some embodiments, the intermittent time period is selected by a user, for example, via the user interface 126. In some embodiments, the intermittent time period can range from 1 second to 60 seconds, more particularly from 1 second to 30 seconds and even more particularly from 1 second to 10 seconds.

At step 490, a portion of battery power at the ultrasound imaging device 112 is prioritized for activating the short-range wireless connection for initiating location tracking while the ultrasound imaging device 112 is in the inactive mode. In some embodiments, software 154A is configured for prioritizing a portion of battery power at the ultrasound imaging device 112 for activating the short-range wireless connection while the ultrasound imaging device 112 is in the inactive mode. In some embodiments, the portion of battery power at the ultrasound imaging device 112 to be prioritized for activating the short-range wireless connection is a fixed portion of the battery power. In some embodiments, the portion of battery power at the ultrasound imaging device 112 to be prioritized for activating the short-range wireless connection is a selected percentage of the remaining battery power at the ultrasound imaging device 112. In some embodiments, the portion of battery power at the ultrasound imaging device 112 to be prioritized for activating the short-range wireless connection is selected by a user, for example, via the user interface 126.

In some embodiments, method 300 further includes transmitting one or more beacon signals for initiating location tracking of the ultrasound imaging device 112. In some embodiments, the one or more beacon signals is transmitted through at least one of an Apple™ (e.g., “Find My”) and an Android™ (e.g., “Find My Device”) finding protocol. In some embodiments, the one or more beacon signals is transmitted to one or more beacons and/or receivers within a third-party network, such as a hospital network.

In some embodiments, transmitting one or more beacon signals for initiating location tracking of the ultrasound imaging device 112 includes transmitting the one or more beacon signals to a workflow application at the mobile device 110. In some embodiments, a user can trigger the transmitting of one or more beacon signals for initiating location tracking of the ultrasound imaging device 112 via the workflow application at the mobile device 110. For example, the user can send a request, via the workflow application, to transmit one or more beacon signals for initiating location tracking of the ultrasound imaging device 112.

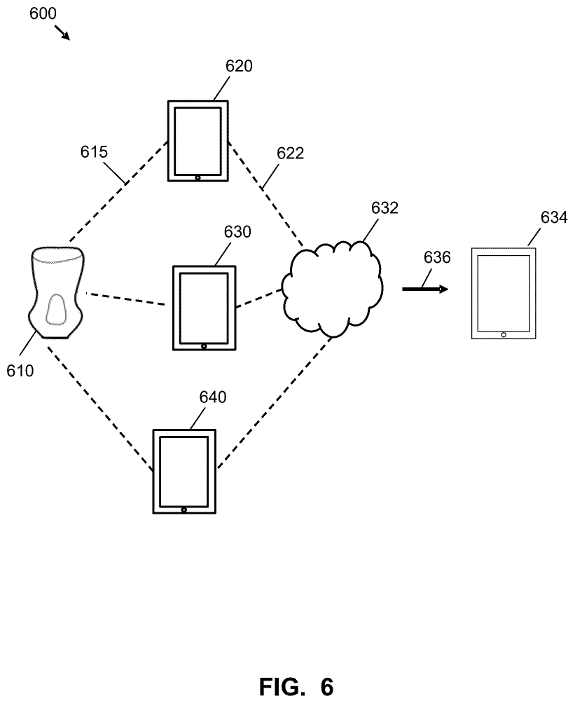

At least one embodiment of the present invention leverages the power of crowdsourced data to locate an ultrasound imaging device 112. FIG. 6 is illustrative of a system within the scope of one embodiment of the present invention, for deployment of an Apple™ (e.g., “Find My”) and an Android™ (e.g., “Find My Device”) finding protocol, to determine a location of an ultrasound imaging device 112 using crowdsourcing features, such system generally indicated at 600. A benefit of using short range radio communication protocols (such as Bluetooth) to track an ultrasound imaging device 610 is the ease of compatibility with “intermediary” tracking devices such as mobile devices 620, 630 and 640 (which may be third party mobile devices such as smartphones, tablets, etc . . . ). Tracking device signal data 615, captured through devices 620, 630 and 640, via a mobile application (mobile app) may be transferred to cloud 632, via communications network 622 for processing. In one embodiment, when ultrasound imaging device 610 is misplaced or stolen, a user of multi-purpose electronic device 634 can log into their finding account (for example an iCloud account on the Find My website, the Find My app on another Apple device, or a Google account on the Find My Device network—in cloud 632, via communications network 636). This allows the user, via multi-purpose electronic device 634, to see the approximate location of the missing ultrasound imaging device 610, such as on a map, displayed on a screen interface of multi-purpose electronic device 634. In this way, location tracking works by using one or more of GPS, Wi-Fi, and cellular signals to pinpoint the whereabouts of ultrasound imaging device 610.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

The system of the present invention uses a transducer (e.g., a piezoelectric or capacitive device operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer.

In various embodiments, a multi-purpose electronic devices/display devices may be, for example, a laptop computer, a tablet computer, a desktop computer, a smart phone, a smart watch, spectacles with a built-in display, a television, a bespoke display or any other display device that is capable of being communicably connected to an ultrasound imaging device/probe. Multi-purpose electronic devices/display devices may host a screen (such as shown in FIGS. 1 and 2), and may include a processor, which may be connected to a non-transitory computer readable memory storing computer readable instructions, which, when executed by the processor, cause the display device to provide one or more of the functions of the system (such system comprising at least one multi-purpose electronic device and at least probe). Such functions may be, for example, the receiving of ultrasound data that may or may not be pre-processed; scan conversion of received ultrasound data into an ultrasound image; processing of ultrasound data in image data frames; the display of a user interface; the control of a probe and the display of an ultrasound image on the screen. Such a screen may comprise a touch-sensitive display (e.g., touchscreen) that can detect a presence of a touch from the operator on screen and can also identify a location of the touch in screen. The touch may be applied by, for example, at least one of an individual's hand, glove, stylus, or the like. As such, the touch-sensitive display may be used to receive an input, for example, indicating the presence or absence of text or annotations on an image. The screen and/or any other user interface may also communicate audibly. Multi-purpose electronic devices/display devices may be configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Also stored in the computer readable memory within the multi-purpose electronic devices/display devices may be computer readable data which may be used by processors within multi-purpose electronic devices/display devices, in conjunction with the computer readable instructions within multi-purpose electronic devices/display devices, to provide the functions of the system. Such computer readable data may include, for example, settings for ultrasound probe, such as presets for acquiring ultrasound data and settings for a user interface displayed on screens. Settings may also include any other data that is specific to the way that the ultrasound probe operates or that multi-purpose electronic devices/display devices operate.

### D. Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.
Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

To aid the Patent Office and any readers of any patent issued on this application in interpreting the claims appended hereto, applicant wishes to note that they do not intend any of the appended claims or claim elements to invoke 35 U.S.C. 112 (f) unless the words “means for” or “step for” are explicitly used in the particular claim.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

1. A method of configuring an ultrasound imaging device to be trackable with short-range wireless connection during an active mode and an inactive mode, the method comprising:
during the active mode, applying one or more data signals being communicated by the short-range wireless connection for location tracking; and
while in the inactive mode, intermittently engaging the short-range wireless connection for initiating location tracking of the ultrasound imaging device.

2. The method of claim 1, wherein intermittently engaging the short-range wireless connection to initiate location tracking of the ultrasound imaging device while in the inactive mode comprises:
detecting a movement at the ultrasound imaging device; and
in response to detecting the movement, activating the short-range wireless connection to initiate location tracking during the inactive mode.

3. The method of claim 2, wherein detecting the movement at the ultrasound imaging device comprises:
receiving a motion signal from a motion sensor at the ultrasound imaging device, the motion signal being indicative of the ultrasound imaging device moving away from a receiving device.

4. The method of claim 2, wherein the ultrasound imaging device moves in recognizable patterns in the active mode and wherein detecting the movement at the ultrasound imaging device comprises:
receiving a motion signal from a motion sensor at the ultrasound imaging device, the motion signal being indicative of the ultrasound imaging device moving in a non-recognizable pattern.

5. The method of claim 2, wherein detecting the movement at the ultrasound imaging device comprises:
receiving a motion signal from a motion sensor at the ultrasound imaging device;
comparing the movement at the ultrasound imaging device to a set of pre-determined movements which correspond to the active mode, for initiating location tracking of the ultrasound imaging device; and
if the movement at the ultrasound imaging device does not match at least one of the pre-determined movements, activating the short-range wireless connection to initiate location tracking.

6. The method of claim 1, wherein intermittently engaging the short-range wireless connection to initiate location tracking of the ultrasound imaging device while in the inactive mode comprises:
intermittently operating a tracking trigger at the ultrasound imaging device to activate the short-range wireless connection to initiate location tracking while the ultrasound imaging device is in the inactive mode.

7. The method of claim 6, wherein intermittently engaging the short-range wireless connection to initiate location tracking of the ultrasound imaging device while in the inactive mode comprises:
prioritizing a portion of battery power at the ultrasound imaging device for activating the short-range wireless connection for initiating location tracking while the ultrasound imaging device is in the inactive mode.

8. The method of claim 1 further comprises:
transmitting one or more beacon signals for initiating location tracking of the ultrasound imaging device.

9. The method of claim 8 further comprises:
transmitting the one or more beacon signals to a workflow application at a mobile device.

10. The method of claim 8 further comprises:
transmitting one or more beacon signals for initiating location tracking of the ultrasound imaging device through at least one of an Apple™ and an Android™ finding protocol.

11. The method of claim 1, wherein the short-range wireless connection comprises one of a Bluetooth™ and a Zigbee™ connection.

12. The method of claim 1, wherein the short-range wireless connection comprises a Bluetooth™ connection which additionally applies one or more data signals to pair the ultrasound imaging device with a mobile device.

13. The method of claim 1, wherein the active mode comprises an operating mode during which the ultrasound imaging device is in use.

14. The method of claim 1, wherein the inactive mode comprises one or more operating modes during which the ultrasound imaging device is in one of hibernation or sleep.

15. An ultrasound imaging device configurable to be trackable with short-range wireless connection during an active mode and an inactive mode, the ultrasound imaging device comprising:
a short-range wireless connection module; and
a processor in communication with the short-range wireless connection module, the processor operable to:
during the active mode, apply one or more data signals being communicated by the short-range wireless connection module for location tracking; and
during the inactive mode, intermittently engage the short-range wireless connection module for initiating location tracking of the ultrasound imaging device.

16. The ultrasound imaging device of claim 15, wherein the processor is further operable to:
detect a movement at the ultrasound imaging device; and
in response to detecting the movement, activate the short-range wireless connection module to initiate location tracking during the inactive mode.

17. The ultrasound imaging device of claim 16, wherein the ultrasound imaging device comprises a motion sensor and the processor is further operable to:
receive a motion signal from the motion sensor, the motion signal being indicative of one or more of:
the ultrasound imaging device moving away from a receiving device;
the ultrasound imaging device moving in a non-recognizable pattern, wherein the ultrasound imaging device moves in recognizable patterns in the active mode; and
the movement being non-matching to a set of pre-determined movements which correspond to the active mode, wherein:
the movement at the ultrasound imaging device is compared to the set of pre-determined movements, for initiating location tracking of the ultrasound imaging device, and
if the movement at the ultrasound imaging device does not match at least one of the pre-determined movements, the short-range wireless connection module is activated to initiate location tracking.

18. The ultrasound imaging device of claim 15, the processor further operable to:
intermittently operate a tracking trigger of the ultrasound imaging device to activate the short-range wireless connection module to initiate location tracking while the ultrasound imaging device is in the inactive mode.

19. The ultrasound imaging device of claim 18, the processor further operable to:
prioritize a portion of battery power at the ultrasound imaging device for activating the short-range wireless connection module for initiating location tracking while the ultrasound imaging device is in the inactive mode.

20. A non-transitory computer readable medium for configuring an ultrasound imaging device to be trackable with short-range wireless connection during an active mode and an inactive mode, the medium comprising computer-readable instructions, which, when executed by one or more processors on the ultrasound imaging device, configures the ultrasound imaging device to:
during the active mode, apply one or more data signals being communicated by the short-range wireless connection for location tracking; and
while in the inactive mode, intermittently engaging the short-range wireless connection for initiating location tracking of the ultrasound imaging device.
